(19) United States
(12) Patent Application Publication
(10) Pub. No.: US 2025/0391521 A1
(43) Pub. Date: Dec. 25, 2025

(54) CROSS-ENTERPRISE DOCUMENT SHARING (XDS) METADATA MORPHING

(71) Applicant: Hyland Software, Inc., Westlake, OH (US)

(72) Inventors: Souvik Sengupta, Kolkata (IN); Arindam Pal, Kolkata (IN); Otto Hunter Gasser, Birmingham, AL (US)

(21) Appl. No.: 18/753,075

(22) Filed: Jun. 25, 2024

(51) Int. Cl. G16H 10/60; G16H 30/20

(52) U.S. Cl. CPC G16H 10/60; G16H 30/20

(57) ABSTRACT

Techniques, described herein, enable a system of a healthcare enterprise to receive a query for Cross-Enterprise Document Sharing (XDS) content in a first document repository. The system can search the first document repository for the XDS content based on the query, and intercept XDS content results of the XDS content based on metadata of the XDS content satisfying the query before being provided in response to the query. The system morphs at least one attribute associated with the metadata of the first set of XDS content in transit before providing at least one of the first set of XDS content results to a graphical user interface or a second document repository.

### FIELD

This disclosure relates to computerized networks and data management systems for morphing cross-enterprise document sharing (XDS) data in an XDS format particularly in the healthcare field.

### BACKGROUND

Computerized networks and data management systems can include a variety of systems, devices, and technologies to enable users to create, store, access, and distribute information. Such networks can include one or more wired networks, wireless networks, or a combination thereof. Each network can include a broad range of interconnected devices, each comprising hardware, software, virtualization technology, etc., which enables the devices to send, receive, process, and/or store information. Examples of such devices can include mobile user devices (e.g., cell phones, tablet computers, laptop computers, etc.) stationary devices (e.g., desktop computer, servers, etc.), and network components and devices (e.g., network hubs, routers, base stations, satellite systems, etc.).

Digital imaging and communications in medicine (DICOM) is a standard used for the communication and management of medical imaging information and related data. DICOM can be used by, for example, hospitals, doctor offices, government agencies, research institutions, and other types of organizations. DICOM can be implemented for storing and transmitting medical images, enabling the integration of medical imaging devices such as scanners, servers, workstations, printers, network hardware, and picture archiving and communication systems (PACS) from multiple manufacturers.

Integrated care continues to emerge as an appropriate service delivery model to provide a safer and higher-quality patient experience. The traditional hierarchical and soloed approach, driven by the missions of single organization or department objectives should continue to evolve toward integrated pathways in the healthcare enterprise. Information sharing can improve the quality of care. From a healthcare information systems point of view, new approaches to data aggregation, storage and sharing is needed. Healthcare enterprises will need to look for solutions based on interoperability standards such as DICOM or HL7, tested on profiles developed by Integrating the Healthcare Enterprise (IHE).

### BRIEF DESCRIPTION OF THE DRAWINGS

The present disclosure will be readily understood and enabled by the detailed description and accompanying figures of the drawings. Like reference numerals can designate like features and structural elements. Figures and corresponding descriptions are provided as non-limiting examples of aspects, implementations, etc., of the present disclosure, and references to “an” or “one” aspect, implementation, etc., does not necessarily refer to the same aspect, implementation, etc., and can mean at least one, one or more, etc.

FIG. 1 is a diagram of an example overview according to one or more implementations (aspects) described herein.

FIG. 2 is a diagram of an example system according to one or more implementations described herein.

FIG. 3 is an example study description for morphing XDS document content according to one or more implementations described herein.

FIG. 4 is an example record with patient ID for morphing XDS document content according to one or more implementations described herein.

FIG. 5 is an example morphing component for morphing XDS document content according to one or more implementations described herein.

FIG. 6 is another example morphing component for morphing XDS document content according to one or more implementations described herein.

FIG. 7 is a diagram of an example system for morphing XDS document content according to one or more implementations described herein.

FIG. 8 is a diagram of an example process flow for morphing XDS document content according to one or more implementations described herein.

FIG. 9 is a diagram of example components of a device that can be used within environments or implementations (aspects) herein.

### DETAILED DESCRIPTION

The following detailed description refers to the accompanying drawings. Like reference numbers in different drawings can identify the same or similar features, elements, operations, etc. Additionally, the present disclosure is not limited to the following description as other implementations can be utilized, and structural or logical changes made, without departing from the scope of the present disclosure.

As utilized in this disclosure, terms “component,” “system,” “interface,” and the like are intended to refer to a computer-related entity, hardware, software (e.g., in execution), and/or firmware. For example, a component can be a processor (e.g., a microprocessor, a controller, or other processing circuitry or device), a process running on a processor, a controller, an object, an executable, a program, a storage device, a computer, a tablet PC and/or a user equipment (e.g., mobile phone, etc.) with a processing device. By way of illustration, an application running on a server and the server can also be a component. One or more components can reside within a process, and a component can be localized on one computer and/or distributed between two or more computers. The term “circuitry” may refer to, be part of, or include an Application Specific Integrated Circuit (ASIC), an electronic circuit, a processor (shared, dedicated, or group), or associated memory (shared, dedicated, or group) operably coupled to the circuitry that execute one or more software or firmware programs, a combinational logic circuit, or other suitable hardware components that provide the described functionality. In some embodiments, the circuitry may be implemented in, or functions associated with the circuitry may be implemented by, one or more software or firmware modules. In some aspects, circuitry may include logic, at least partially operable in hardware.

For healthcare enterprises to achieve interoperability for ensuring access to information, collaboration and workflow management, profiles can be developed for integration. Integrating the Healthcare Enterprise (IHE) has developed various profiles in healthcare enterprises or groups of healthcare organizations (also referred to in IHE as an “Affinity Domain”) that want to share patient clinical documents and images including: Cross-Enterprise Document Sharing (XDS), Patient Identifier Cross-Reference (PIX), and Patient Demographics Query (PDQ), for example. IHE demand seamless sharing of healthcare information between computer systems across healthcare organizations (e.g., entities or enterprises) irrespective of the vendors. XDS is one such IHE profile that manages the sharing of documents between healthcare enterprises. XDS defines metadata objects and their attributes using classes provided by a specification (e.g., OASIS ebXML RegRep 3.0, or other repository specification). For example, ebRIM (Electronic Business using extensible Markup Language) Registry and Repository (RegRep) specification describes a way to implement registry and repository servers and clients using standard interfaces, protocols and an information model for publishing, management, discovery and retrieval of arbitrary content and metadata that describes it. IHE highly constrains the use of the ebXML RegRep parts of the specification.

An XDS profile is an access protocol that allows healthcare documents from different systems to be shared over a network of healthcare providers delivering healthcare. XDS provides specifications or standards for managing the sharing of documents between healthcare enterprises or organizations of the network by facilitating registration, distribution, and access of patient information and associated documents, which is done by defining common methods for information technology security, patient information integrity, and patient ID management.

XDS can be configured to support document special demands for images of digital imaging and communications in medicine (DICOM) standard, for HL7, and other healthcare standards and formats. In particular, the DICOM standard describes a format of medical images, as well as their distribution, in terms of a communication protocol. The information model for DICOM can include one to many relationships between main information entity levels, which include a patient, a study, a series and an instance/image, in which a core unit is an imaging study. Each DICOM instance or DICOM object can be in a binary format and can consist of a header, which can include a dictionary of key value pairs. A key can also be referred to as a DICOM tag that are associated with a DICOM dictionary of tags.

An aspect of DICOM systems can include indexing and searching operations of DICOM metadata or metadata of other formats (e.g., XDS, HL7, or other format), whereby medical records of a DICOM study formatted according to the DICOM standard can be indexed by extracting metadata from the header of each DICOM object in order to properly persist the medical studies, as well as support radiology and other medical study (multi-ology) workflows. In particular, a clinician can direct viewable studies across volumes of metadata for analysis and analytics in an efficient and simplified manner of performing medical studies according to indexing and searching operations between different healthcare archives of different formats (e.g., XDS, HL7, DICOM, or other format).

Metadata may refer to information regarding the content (e.g., DICOM and/or non-DICOM content). Metadata can provide information regarding the content such as, for example, information about a DICOM image data including dimensions, size, modality used to create the data, bit depth, and settings of the medical imaging equipment used to capture a DICOM image. Other content such as XDS content or content of another format can provide patient information related to the content, including information, for example, a patient's medical history, file, demographics, immunization status, radiology images, medical allergies, basic patient information (e.g., age, weight, or other patient related information), vital signs or billing information. Alternatively, or additionally, other non-DICOM content can include medical image data objects such as, for example, diagnostic objects having standard object formats such as, JPEG, PDF, MPEG, TIFF, WAV, but that are not DICOM objects. Such content may also be objects having no standard information format or model so that the associated data format does not specify required and/or standard identifying information that is associated with the content.

Content can refer to an XDS content, a DICOM content or content of other formats (e.g., HL7) including a medical record or file, which can include a header and a body arranged according to a format specified by DICOM, XDS or other standards. The header in a DICOM format, for example, can include a variety of attributes (also referred to as keys, tags, or DICOM tags) in DICOM standards, some of which can be identifying information. Attributes of metadata, in particular, can include data or content that associates the content with a resource or patient, for example. Attributes of metadata can describe a functionality, extend a tag, or have a value associated with an attribute ID, for example. The body of can include, for example, the imaging information of the corresponding X-ray, MRI, etc., which can also include pixel data as a part of DICOM metadata. Indexing and searching operations herein can include values of tags of the DICOM headers of a DICOM object of every medical imaging study or DICOM study, where implementations/aspects herein enable simplified searching of the indexed DICOM metadata by morphing of metadata for content (e.g., XDS content) to more closely align with DICOM or other content formats of different healthcare enterprises and vice versa based on whether the content is incoming or outgoing in the system.

The quantity of records to be indexed and searched in a given batch or set of medical records or DICOM studies can be large and complex (e.g., involving up to or more than millions of records, multiple studies, or across multiple institutions). Each DICOM medical study can involve one or more various modality inputs and outputs of DICOM objects as well as past and present cross-discipline studies on a patient, patient ID, a particular health record, a timeline, medical specialty, medical enterprise, topic, etc., that can span a patient, a study, a series, an instance, other attribute/information entity level of the metadata associated with a document or file as DICOM content.

XDS enables users (document consumers) to retrieve different types of documents (letters, results, images, and folders) in a quick and consistent way. XDS allows handling of any type of content so that each document is viewed in its original form. XDS thus provides a foundation on which to build virtual longitudinal patient record on the fly from a variety of clinical documents created by different organizations.

However, instances may arise in an XDS document sharing workflow when a document is submitted for storage or is queried to an XDS system that certain document/patient metadata/attributes of metadata can be missing or incorrect for various reasons. Additionally, some third party XDS actors or components of an enterprise healthcare system may lack the functionality to map patient IDs associated with the content to the correct domain. In those situations, it is desirable for interoperability and data integrity so that the XDS system of a healthcare enterprise can have mechanisms to automatically transform existing Document/Patient metadata in XDS transaction requests and responses, especially in transit before the requests or responses arrive to a destination (e.g., the XDS repository, a graphical user interface, a DICOM repository or a repository of a format that is different from an XDS format). Customers or clients could also demand that this automated metadata morphing functionality exist in XDS similarly as in DICOM vendor neutral archives (VNAs) or other formats of other vendors. IHE does not define a XDS profile to update metadata or attributes of metadata in transit. Instead, an update of all stored metadata is defined by a Metadata Update Profile where a new version of the document metadata is retrieved after being submitted for replacing or modifying the relevant existing old metadata that is being stored. This Metadata Update Profile is intended to be executed manually on an as needed basis by an Administrator, which can be inefficient to a user and provide a less than desired quality of experience for a healthcare enterprise.

In aspect, a healthcare enterprise system can receive a query for a first set of Cross-Enterprise Document Sharing (XDS) content in a first document repository for XDS content. The first document repository can be an XDS repository in an XDS profile or another healthcare enterprise system, for example. The system searches the first document repository for the XDS content based on the query, and intercepts XDS content results based on the XDS metadata of the XDS content satisfying the query before being provided to another document repository of another format, or to the user in a user graphical interface in response to the query. The system can operate to dynamically transform existing document/patient metadata in XDS transaction requests and responses according to predefined rules of the healthcare enterprise or formatting for the second document repository of a different format. For example, the system can operate to morph one or more attributes associated with the metadata of XDS content in transit before providing the XDS content results to a graphical user interface or the second document repository, which may be a DICOM VNA or other document repository of content in another format (e.g., a DICOM format, HL7, XDS, or other format).

In another aspect, the healthcare enterprise system can morph XDS content intercepted in transit based on a user request or an XDS transaction request and an XDS transaction request by modifying a study description of the XDS content to be compatible or user friendly for another healthcare system or another document repository that includes content in a different format (e.g., DICOM, HL7, or other format) than XDS content. For example, the system can operate via processing circuitry (e.g., one or more processors or firmware of software and hardware) to remove and morph at least a portion (e.g., a prefix or suffix) of a study description of the XDS content based on criteria of another document repository or another healthcare enterprise. An updated string can then be used as an EventCode (a code that is XML encoded) for one or more stored key object selection (KOS) DICOM documents or documents of another format.

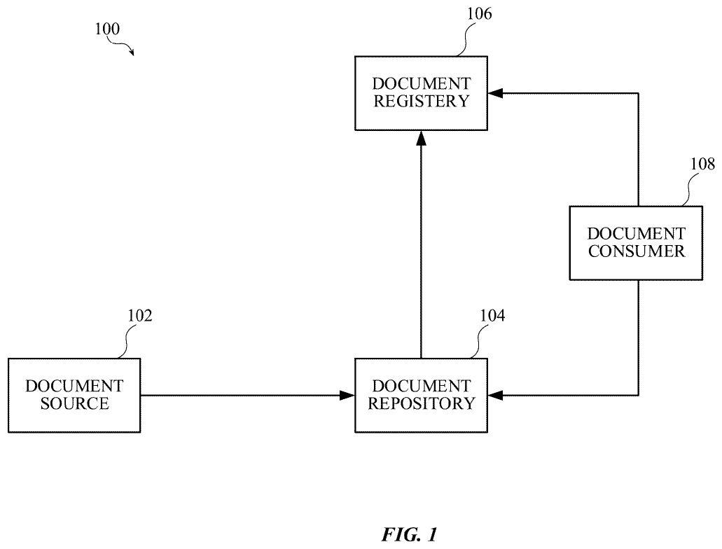

FIG. 1 illustrates an example system 100 of an XDS profile. The system 100 comprises various actors as components that include a document source 102, a document repository 104, a document registry 106 and a document consumer 108, each having inputs/outputs, respectively.

The document source 102 is coupled to the document repository 107, and configured to produce and publish documents (or content), and further sends or submits the content to the document repository 104. The document source 102 can further supply metadata of the content to the document repository 104 for subsequent registration of the metadata and content with the document registry.

The document repository 104 is coupled to the document registry 106 and the document consumer 108. The document repository 104 is configured to provide persistent storage of the metadata and content. The document repository 104 is configured to register the documents (or content) with the document registry 106 and assigns an appropriate unique ID (e.g., a patient ID) to the metadata and content for subsequent retrieval by the document consumer 108. The document repository 104 can store documents in a number of different formats (e.g., AVI, CDA, PDF, or other format) as well as an XDS format.

The document registry 106 is coupled to the document repository 104 and to the document consumer 108. The document registry 106 is configured to maintain metadata associated with each registered document or content in a document entry and further enforces various healthcare policies at the time of registration. The document registry 106 further generates a link to access where the content or document is stored in the document repository 104. The document registry 106 can further respond to queries from the document consumer 108 with XDS content results satisfying criteria of the query.

The document consumer 108 is coupled to the document repository 104 and to the document registry 106. The document consumer 108 is configured to query the document registry 106 for any content satisfying any criteria associated the query, and provide the content or the document from the document repository 104 in a user display or graphical user interface.

In various aspects, the system 100 can be operable among or within various proprietary components that function to transfer and store documents including associated metadata. When various vendor components communicate among each other by transferring and storing content in various transactions performed by the document source 102, the document repository 104, the document registry 106 and the document consumer 108, for example, there arises a possibility of format agreement matching among various formats of the content. As such, instances may arise in an XDS document sharing workflow as in system 100 when content or metadata is submitted for storage or is queried to an XDS system that certain document/patient metadata/attributes of metadata can be missing or incorrect. For example, components of an enterprise healthcare system may lack the functionality to map patient IDs associated with the content to a correct domain or link. Therefore, components are desired to dynamically transform existing metadata in XDS transaction requests and responses in transit before the requests or responses arrive to a destination (e.g., the XDS repository, a graphical user interface, a DICOM repository or a repository of a format that is different from an XDS format) from the document consumer 108.

In an aspect, one or more components can operate to intercept content and metadata that satisfies a query before the content satisfying the query is provided to another document repository of proprietary content (e.g., DICOM, HL7, or other content). In response to being intercepted in transit an attribute of metadata or metadata of the result to the query or a query response can be morphed or transformed according to various rules. Likewise, XDS requests for documents can also be morphed so that the attribute of the metadata or the metadata in the requests and in responses from the components of system 100 can be transformed according to a set of rules.

For example, descriptions or study descriptions of content or metadata (e.g., a medical study, a medical resonance imaging (MRI) image, or other medical patient information) can be morphed to remove or modify portions of the attributes of the metadata or the metadata. For example, a suffix in the study description can be removed or updated. This updated string can then be used as an XDS EventCode for the content that is being stored in a different format in a same or different repository. This EventCode can then be used for retrieving, providing, or storing the content, for example, as one or more KOS DICOM documents.

In another example, patient IDs can be morphed by removing or adding insignificant digits or alpha-numeric symbols according to an incoming format. For example, leading or preceding zeros of a patient ID can be removed. Alternatively, or additionally, tailing or proceeding zeros could be removed where they are insignificant or added to match a length requirement/threshold of the patient ID or other qualification of a different formatting than the healthcare enterprise managing the content and its associated metadata.

In yet another example, patients of a healthcare enterprise could have multiple IDs. Upon requesting or retrieving information associated with one patient ID, either of the same healthcare enterprise or a different one of an XDS content, the system 100 can morph the incoming/outgoing patient ID to one or more of the same patient's other IDs.

Moreover, in another example, the system can also be configured to query with a Patient Identifier Cross-Reference (PIX) query to look up the Affinity Domain patient ID or returned patient ID that is morphed in the query results. In this manner, an XDS consumer or the document consumer 108 can be able to query beyond a local patient ID, for example.

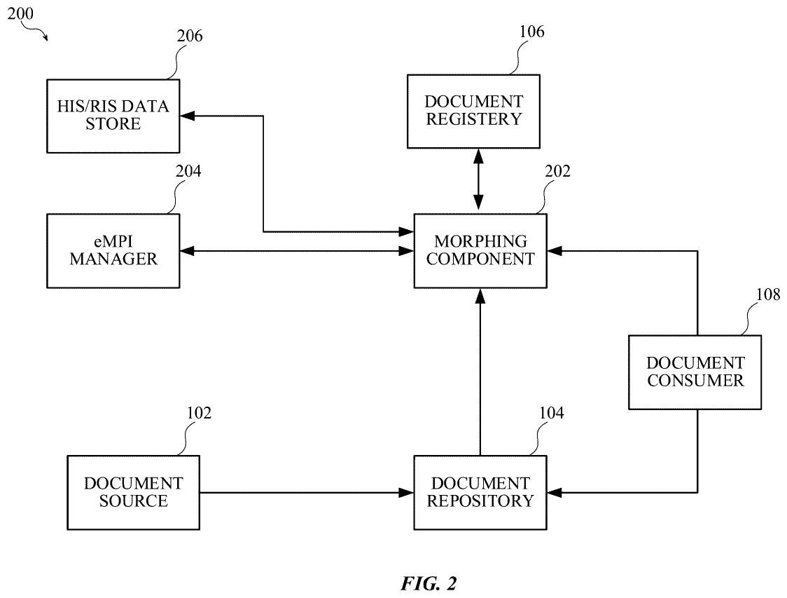

FIG. 2 is another example system 200 of an XDS profile. System 200 includes similar components as system 100, additionally including a morphing component 202, an enterprise master patient index (eMPI) manager or patient manager 204, and a hospital information system (HIS)/radiology information system (RIS) 206. The number of devices and/or network, illustrated in FIG. 2, is provided for explanatory purposes only. In practice, there can be additional devices, components and/or networks, fewer devices, components and/or networks, different devices, components and/or networks, or differently arranged devices, components and/or networks than illustrated in FIG. 2. Devices and components of system 200 can interconnect via wired connections, wireless connections, or a combination of wired and wireless connections. Also, in some implementations, one or more of the devices or components of system 200 can perform one or more functions described as being performed by another one or more of the devices or components of system 200.

The morphing component 202 comprises inputs/outputs coupled to inputs/outputs of the document registry 106, the document consumer 108, the document repository 104, the patient manager 204, and the HIS/RIS data store 206. The morphing component 202 is coupled to the document registry 106, the document consumer 108, the patient manager 204 and the HIS/RIS data store 206.

The morphing component 202 is configured to intercept XDS transaction requests and responses, and transform the content of documents/patient metadata in the XDS transactions based on one or more rules/actions. A transaction or an XDS transaction can refer to as an exchange of information between the actors or components in a healthcare enterprise or system using messages based on established standards of the IHE Technical Framework. Some of the XDS transactions that can be intercepted by the morphing component 202, for example, include, but are not limited to, a registry stored query request, a registry stored query response, a register document set, a retrieve value set, a multi-patient stored query request, a multi-patient stored query response, an update document set, a register on demand (RegisterOnDemand) document entry, or a delete document set, in which each can correspond to a response/a request message.

In particular, the registry stored query request/response is used by the document registry 106 and document consumer 108 for a set of pre-defined queries that include document entry objects by patient ID for an interval of time, document type, medical practice setting, or author, for a folder, for a contents in a folder or a submission set by time of submission; these queries return metadata for one or more types of registry objects or object references for one or more types of registry object. The register document set can be used to register a set of document-associated metadata (e.g., in the document registry 106). The retrieve value set can be used to retrieve a value set (as a request/response) for an object identifier as it relates to a consumer (or patient) and content (or document) in the document repository 104. The multi-patient stored query is used for a variety of queries for multiple patients to retrieve metadata or object references associated with multiple patients.

The morphing component 202 can be configured to perform morphing of incoming XDS messages as a request or outgoing XDS messages as a response, for example, or vice versa. The morphing component 202 can perform such transformations based on user customizable extensible style sheet transformation (XSLT) code for XDS Subjective Objective Assessment and Plan (SOAP) requests and response Extensible Markup Language (XML).

Alternatively, or additionally, the morphing component 202 can be configured to follow a rule based object morphing for transforming XDS message requests/responses from one formatting to another before being presented to a user interface (e.g., a graphical user interface) or another healthcare enterprise system component (e.g., another document repository or registry). The morphing component 202 is configured to perform transformations of XDS responses/requests in transit from one healthcare enterprise system/component to another, or within components of a same healthcare enterprise system. The set of rules can be configurable by a user or customized selection, or predefined as actions to be performed for transformation in transit to match formatting (e.g., a DICOM, HL7, or other format) of a healthcare enterprise system.

In some examples, the rules for transformation XDS message requests/responses performed by the morphing component 22 can include actions that include updating any of the XDS document metadata or corresponding attributes within an XDS message request/response with a new value by replacing the older one. Additionally, or alternatively, a prefix of a string (e.g., an alpha-numeric, or other symbol string of a name or code) could be added or removed, or a suffix could be added or removed. In another example, the morphing component 202 could perform a RegexReplace function that replaces at least a portion or all of a string with another string or portion of a string. The morphing component 202 can also be configured to trim a string of an XDS message request/response, associate or generate sub-string thereto, or correct an incorrect value using a Regex Mismatch function, for example, in order to transform the XDS request/response for content or metadata to a different pattern or value format.

The eMPI manager or patient manager 204 is configured to manage patient information identity or patient IDs across the components or systems in order to ensure that when sharing a patient document as a message response to a request the same or correct patient is associated with the proper content or document. The eMPI manager 204 can be configured in the system using either of PIX Manager or PDQ Consumer profile details, which the eMPI manager 204 uses to manage patient IDs and create patient IDs. A PIX profile supports the linking of patient identifiers from multiple identity domains. PDQ profile supports the ability to search for patients' documents; it allows querying by a minimal set of demographics and getting in response a complete set of demographics, usually including patient identifiers in domains of interest. The morphing component 202 queries the configured eMPI source to the eMPI manager 204 either by using the Patient Id (or any of the patient details in case of PDQ transaction i.e.—Name, DOB, Gender, Address) to get the other required patient details for morphing content, metadata or its attributes.

The HIS/RIS data store 206 can be configured as a database with database server instance names associated with content or documents of patients. The morphing component 202 can query the HIS/RIS data store 206 using the patient ID or an accession number as reference to obtain other patient/document details for morphing content of XDS message responses/requests in transit. The morphing component 202 can query the eMPI manager 204 or the HIS/RIS data store 206 to obtain other patient data from different domains that can be applied to XDS metadata.

In aspects, the morphing component 202 can be configured to enable customizable code (e.g., C# code or other code) to be written for more complex morphing logic and load an external dynamic link libraries (DLLs) as a morphing plugin to perform customized transformations of XDS message requests/responses based on formatting of a healthcare enterprise before being provided to a user in a graphical user interface or in a different repository of the enterprise system (e.g., DICOM, HL7, or other format). The morphing component 202 can implement either an XSLT interface as XML input or a rule based interface (XDS metadata object input) to perform transformations of the XDS message responses/requests.

The morphing component 202 can operate to transform descriptions or study descriptions of content or metadata (e.g., a medical study, a MRI image, or other medical patient information) by removing or modifying portions of the attributes or metadata. A suffix in the study description can be removed or updated. This updated string can then be used as an XDS EventCode for the content that is being stored in a different format in a same or different repository. This EventCode can then be used for retrieving, providing, or storing the content, for example, as one or more KOS DICOM documents. A KOS DICOM or DICOM KOS document can be a manifest object (a medical imaging object (e.g., a graphical icon, cursor, or other object) or non-imaging objects (e.g., text or the like)) that is stored into XDS and references/links to a DICOM study resulting from a modality (e.g., an X-ray machine, an MRI, or other imaging device). XDS or XDS for Imaging (XDS-I) defines the mapping of DICOM tag values to XDS metadata attributes such as EventCode.

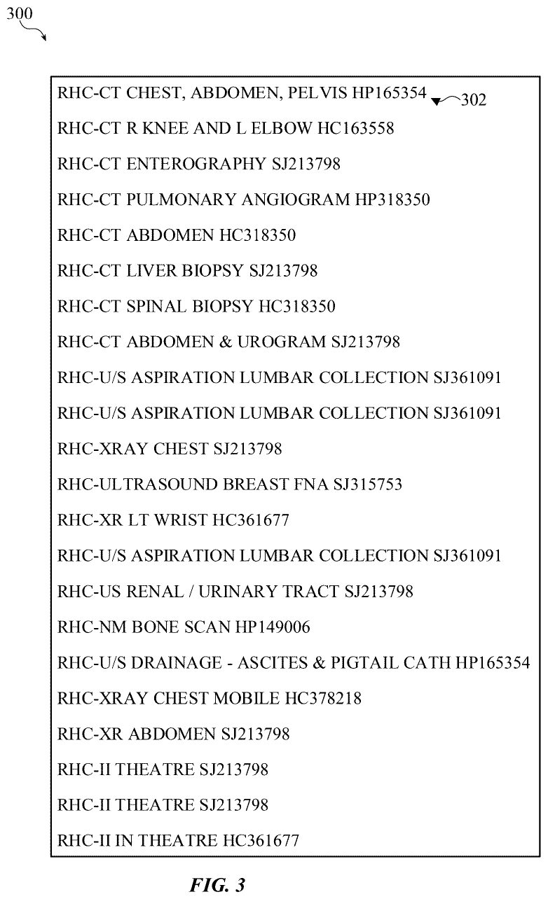

FIG. 3 illustrates an example of study descriptions 300 that can be transformed or morphed by the morphing component 202 of FIG. 2. The morphing component 202 of FIG. 2 can operate to transform a study description in transit to a user graphical interface, the document repository 104 or another repository or data store, for example. The study descriptions 300 can comprise medical study descriptions 300 for one or more patients. For example, the morphing component 202 can transform the study description 302 (e.g., RHC-CT CHEST, ABDOMEN, PELVIS HP165354) or any of the other study descriptions 300 by having a portion or all of the description modified/removed/replace. For example, a suffix of the study description 302 can be morphed, and then an updated string of study description 302 can be used as an EventCode to be used for sending the value or attribute of metadata associated with the content of a document. In this example, the study description 302 has the suffix HP165354, which may not be of significant use in the healthcare enterprise system sending or receiving the XDS message request/response, depending on the vendor. Thus, the morphing component 202 operates to intercept these XDS message requests/responses between the components (e.g., the document registry 106, the document repository 104, and the document consumer 108) and transform the attributes or metadata to a desired format, update wrong values, correct values, or trim portions.

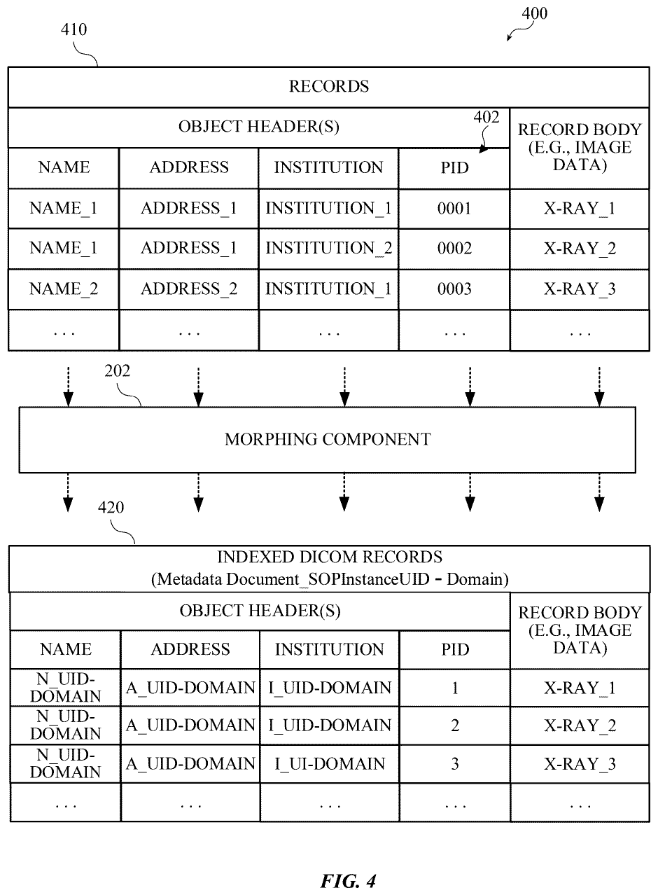

FIG. 4 illustrates an example of study records 410 with a patient ID or PID and other content that can be transformed or morphed by the morphing component 202 of FIG. 2. The patient record content 400 can include study records 410 with XDS objects of a medical study, morphing component 202, and indexed records 420, which can be in a different format (e.g., DICOM, HL7 or otherwise). As described herein, a set (i.e., one or more) of DICOM studies/records can be referred to as a DICOM batch or study, and a DICOM record can be referred to as a DICOM object or instance. Referring to FIG. 4, each content of record 110 can include a record or DICOM object header that includes one or more tags or attributes and a record body. Examples of tags can include patient name (e.g., NAME_1 or NAME_2), patient address (e.g., ADDRESS_1 or ADDRESS_2), institution name (e.g., INSTITUTION_1 or INSTITUTION_2), or other tags. An example of a record or object body can include image or pixel data (e.g., X-RAY_1 or X-RAY_2).

The morphing component 202 can operate to intercept requests or responses to XDS transactions to morph data according to another enterprise formatting. For example, the PIDs of the PID column 402 illustrates example patient IDs in the records 410. The patient IDs of the PID column 402 can be morphed by removing or adding insignificant digits or alpha-numeric symbols according to an incoming format. For example, leading zeros of a patient ID can be removed as indicated in the records 420. Alternatively, or additionally, following or proceeding zeros could be removed where they are insignificant, or these could added to match a length requirement of the patient ID column 402 or other qualification of a different formatting than the healthcare enterprise managing the content and its associated metadata.

A DICOM study, which belongs to a patient can be a collection of DICOM instances. Each DICOM instance can contain business keys that allow a DICOM study to be assembled from the DICOM instances header information. The business keys can include issuer of patient ID/patient ID (or medical record number (MRN)) at a patient level, a study instance unique ID (UID) (studyinstanceUID) at a medical study level, a series instance UID (seriesinstanceUID) at a series level, and a DICOM service object pair (SOP) instance UID (SOPInstanceUID) at a DICOM instance level. The studyUID, seriesUID and SOPInstanceUID can be tags that have an associated value of type UID tags. A patient ID (PatientID)/MRN while usually present do not necessarily have a value according to the DICOM standard. Other important tags can be used in association with a DICOM object as business keys as well. The morphing component 202 can operate to transform any one of the content to a DICOM formatting or other format based on the rules or actions predefined or customized.

Patients of a healthcare enterprise system could have multiple IDs. Upon requesting or retrieving information associated with a patient ID, either of the same healthcare enterprise or a different one of an XDS content, the morphing component 202 can morph the incoming/outgoing patient ID to one or more of the same patient's other IDs. The morphing component 202 can also be configured to query with a Patient Identifier Cross-Reference (PIX) query to look up the Affinity Domain patient ID or returned patient ID that is morphed in the query results or responses. This can enable an XDS consumer or the document consumer 108 to query beyond a local patient ID, for example, or be associated with a mastery patient ID for all patient IDs.

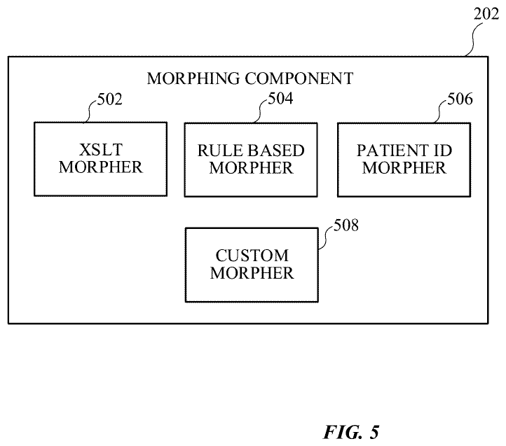

FIG. 5 illustrates an example morphing component with further detail. The morphing component 202 can be a separate component in the system 200 or be co-located inside the registry (e.g., document registry 106). The morphing component 202 includes various components for performing different types of morphing that include an XSLT morpher 502, a rule based morpher 504, a patient ID morpher 506 and a custom morpher 508. When a consumer updates a document, transitions among the other actors or components can occur according to content parameters or a type of morphing. For example, XSLT morphing, rule based morphing, custom morphing, or a use case morphing (e.g., a patient ID morphing) that can be performed by the components including the XSLT morpher 502, the rule based morpher 504, the patient ID morpher 506 and the custom morpher 508, respectively. These morphing functions can include content that is in XDS/XML format, or an IHE formatting in requests/response messages.

For example, XSLT morphing can be performed in the XSLT morpher 502 to transition the XDS content, which is XML based content, according to an XSLT register while the transitions are happening or requests/responses are in transit before being stored in another data store or provided to the user in a graphical user interface. The transformations can be made into a similar format in ITI domain such as in “ebRIM: the “ebXML Registry Information Model version 3.0”, for example. Thus, the XSLT morpher 502 can modify XML objects and update those certain formations from the XMLs.

Using the HIS/RIS data store 206 or source with a patient database, a hospital information system can obtain the patient data from the hospital information system or radio information system database (HIS/RIS Database) and the morphing component 202 morphs certain patient information in the incoming or outgoing planes (requests/responses). In rule based morphing as performed by the rule based morpher 504, the morphing rules for transformation of the XDS content can be global or based on a particular system formatting. The morphing or transformations can be the same for content, metadata or attributes being received in message responses or going out in message requests. The morphing component 202 can intercept either responses or requests to any of the components of system 200, for example, to add, subtract or modify content, metadata or attributes in order to integrate the XDS data with an enterprise system format or formatting (e.g., DICOM, HL7, or other local formatting pattern) for local user operability, quality of experience, ease of use, and familiarity. Thus, the morphing component 202 provides a dynamic functionality in the transit of data transactions with an XDS profile to integrate with different systems of different vendors across content, metadata or attribute inconsistencies and interoperability.

In an aspect, one or more of the components of the morphing component 202 can be selected for operation or not. An administrator working in a DICOM vendor neutral achieve (VNA) system capable of receiving and storing DICOM information and other types of data from a variety of sources, such as, for example, research instructions, hospitals, or doctor offices, may want to transmit stored data on XDS or an XDS component of the system 200. The administrator can then select which functions to enable for morphing by selecting any one or more of the XSLT morpher 502, the rule based morpher 504, the patient ID morpher 506 and the custom morpher 508. This selection can be determined based on whether the administrator (or other user) desires to transform incoming or outgoing XML based content or XDS content with the XSLT morpher 502, use a set of predefined rule based morphing with the rule based morpher 504, certain use cases such as, for example, patient IDs with the patient ID morpher 506, or customize the morphing of content by implementing a DLL code with the custom morpher 508. Each of these components update the content, metadata or attributes, which can be associated with a medical image, in transit before the information is stored or provided to the user or administrator in order to match a particular value or pattern for the content, metadata or attributes from an XDS profile to that of another formatting across healthcare systems.

For example, DICOM comprises a binary protocol involving the use of DICOM tags. In contrast, XDS metadata is XML based, while it is a standard, content, metadata and attributes elements are based in XML. While DICOM has its own binary protocol, XDS is XML based on Simple Object Access Protocol (SOAP) web services and not binary. DICOM has its own protocol that is different. The XSLT morpher 502 can be operable to transform XDS SOAP requests and response XML with user customizable XSLT for any one of the XDS transactions, including, but not limited to, a registry stored query request, a registry stored query response, a register document set, a retrieve value set, a multi-patient stored query request, a multi-patient stored query response, an update document set, a register on demand (RegisterOnDemand) document entry, or a delete document set. Each of the components including the XSLT morpher 502, the rule based morpher 504, the patient ID morpher 506 or the custom morpher 508 of the morphing component 202 can operate to update values, add/subtract prefixes or suffixes, trim content, add/subtract sub-strings, or modify/correct values of a string, metadata, attribute or content, for example.

For example, the XSLT morpher 502 can operate as a component of the morphing component 202 via processing circuitry (e.g., one or more processors or firmware of software and hardware). The XSLT morpher 502 can modify XML objects including the content, metadata or attributes by updating or modifying certain formations from XMLs. The data being morphed can be in XDS/XML format, in IHE formatting, such as XSLT morphing in the XSLT morpher 502 while transitions are happening that are in a similar format in the IHE IT Infrastructure (ITI) domain, such as by using an XDS format for the ebXML Registry Information Model (ebRIM) (e.g., “ebXML Registry Information Model version 3.0” or the like).

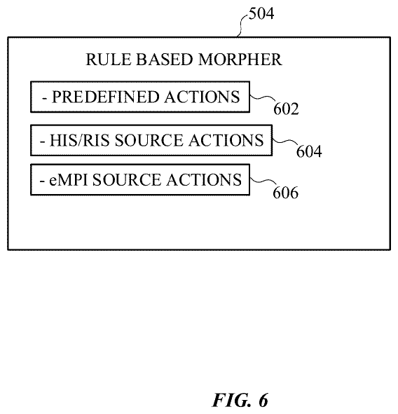

The rule based morpher 504 can operate to match a value or pattern of values/alpha-numeric symbols to another value or pattern, from an XDS content to another formatting or system (DICOM VNA, HL7 protocol or the like), for example. With reference to FIG. 6, the rule based morpher 504 can operate to use a set of predefined actions 602, a set of HIS/RIS actions 604, and a set of eMPI source actions 606. The predefined actions 602 can operate, for example, to trim or add new characters to the content, metadata or attributes by performing a string manipulation. An updated string that has been morphed, for example, can then be used as an EventCode for one or more stored key object selection (KOS) DICOM documents or documents of another format.

The morphing component 202 can also perform rule based morphing using the HIS/RIS actions 604 to morph or transform patient information from the HIS/RIS data store 206 in the incoming and outgoing messages such as to remove, add or morph/modify at least a portion (e.g., a prefix or suffix) of a study description of the XDS content based on criteria of another document repository or another healthcare enterprise; this can enable the study descriptions of strings to be formatted according to various healthcare enterprise preference matches and then be used in a VNA or other vendor specific archive.

The morphing component 202 can also use eMPI source actions 606 as a patient manager tool. The eMPI source actions 606, for example, can be used to query the eMPI/patient manager 204 with a patient ID or obtain a patient ID and morph incoming/outgoing patient IDs to another patient ID. In an example, a patient may visit one healthcare enterprise, receiving one patient ID and then visit another enterprise, receiving another patient ID. The morphing component 202 can use eMPI source actions 606 to act with the eMPI/patient manager 204 to map different patient IDs to one another and the same patient/patient information. Therefore, the morphing component 202 can operate to query from the eMPI, generate/obtain a patient ID mapping, as well as modify one or more patient IDs of a same patient. The modified or mapped patient ID can then be used to obtain documents or content associated with this patient from different healthcare enterprise systems. In ITI standards, for example, a master patient ID and a local patient ID can be used by going to a patient ID cross reference (PIX) manager. The master patient ID is used to provide the ability to query the data stores or other components of the XDS profile in system 100, for example. The morphing component 202 can use the eMPI source actions 606 to intercept the request and form the PIX query with morphing to obtain a correct patient ID to obtain query results or XDS content that satisfy the query as XDS content results. This enables the capability to fill in functionality from the other components and add any missing or required metadata if a patient ID is being sent that is not in the proper domain but still valid as well as replace it in the query results.

Additionally, or alternatively, for example, the morphing component 202 with the patient ID morpher 506 can operate to remove or add preceding (leading) or proceeding (tailing) zeros of a patient ID that can be present to satisfy a length requirement or threshold. In this case, the digits or values may or may not be insignificant within the IHE for administrative purposes, and morphing the patient IDs can save storage space and be more user friendly.

The custom morpher 508 can function to perform any morphing not managed or already functional from the other components of the morphing component 202. For example, different DLLs can be imported by the custom morpher 508 based on a user desired functionality.

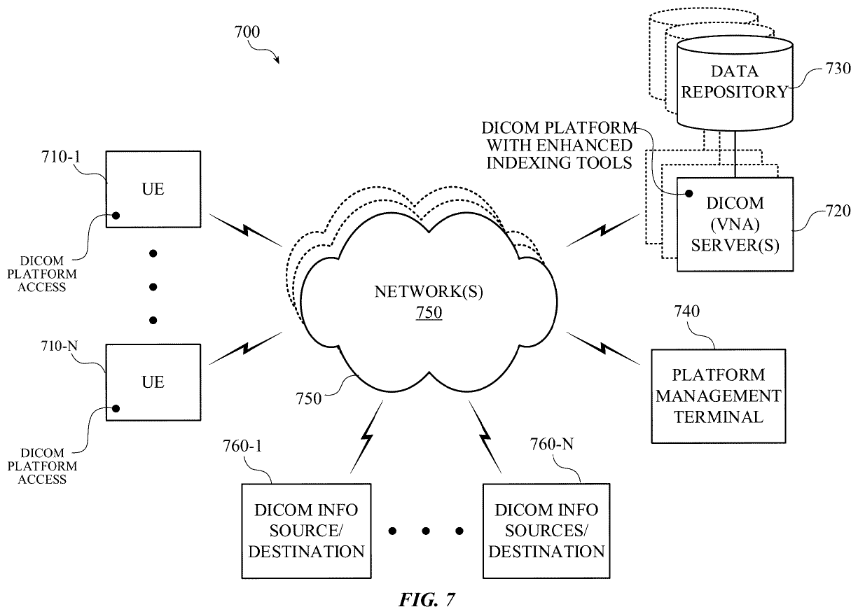

FIG. 7 is a diagram of an example environment 700 in which systems or methods, described herein, can be implemented. As depicted, environment 700 can include user equipment (UE) as medical modalities 710-1, . . . 710-N (where N is greater than or equal to 2 and collectively referred to as “UEs 710”), DICOM servers 720, data repositories 730, platform management terminal 740, DICOM source/destination 760-1, . . . 750-M (where N is greater than or equal to 2 and collectively referred to as “DICOM sources/destinations 760”), and network 750. The number of devices, components and/or network, illustrated in FIG. 7, is provided for explanatory purposes only. In practice, there can be additional devices, components and/or networks, fewer devices, components and/or networks, different devices, components and/or networks, or differently arranged devices, components and/or networks than illustrated in FIG. 7. Devices of environment 700 can interconnect via wired connections, wireless connections, or a combination of wired and wireless connections. Also, in some implementations, one or more of the devices or components of environment 700 can perform one or more functions described as being performed by another one or more of the devices or components of environment 700.

UE 710 can include any type computing device, such as a wired or wireless user device, that is capable of communicating with network 750, such as a medical modality. For example, UE 710 can include a smartphone, tablet computer, laptop computer, or wearable device UE 710 can alternatively include a desktop computer, a radiotelephone, a personal communications system (PCS) terminal (e.g., that can combine a cellular radiotelephone with data processing and data communications capabilities), a personal digital assistant (PDA) (e.g., that can include a radiotelephone, a pager, or Internet/intranet access), or another type of computation or communication device. UE 710 can include any variety of peripheral devices, such as, for example, speaker, cameras, external storage devices, or monitors UE 710 can include a browser or another type of application or interface capable of accessing a DICOM platform hosted by DICOM servers 720.

DICOM servers 720 can include one or more servers or other types of computing devices capable of gathering, processing, searching for, storing, and/or communication information as described herein. In some implementation, DICOM servers 720 can include an application server or a web server that stores one or more applications and/or that permits the one or more applications to be accessed and/or downloaded by UEs 710. DICOM servers 720 can include a single server device, group of server devices, and/or one or more virtual servers. In some implementations, DICOM servers 720 can comprise a DICOM platform as described herein. The DICOM platform can operate, in accordance with DICOM standards, to receive, store, process, secure, and/or provide DICOM information. The DICOM platform can also, or alternatively, include one or more tools, features, or processes capable of perform some or all of the indexing techniques described herein.

In some implementations, DICOM servers 720, in combination with one or more other types of devices, e.g., UEs 710, data repositories 730, or platform management terminals 740, can comprise a DICOM platform. In some implementations, DICOM servers 720 can include a vendor neutral achieve (VNA) system capable of receiving and storing DICOM information and other types of data from a variety of sources, such as, for example, research instructions, hospitals, or doctor offices. In some implementations, DICOM servers 720 can also, or alternatively, be connected to a VNA system and can retrieve DICOM studies and objects from the VNA system for morphing and processing as described herein.

Data repositories 730 can include one or more data storage devices capable of receiving, storing, and providing data related to DICOM information or the management and processing of DICOM information. In some implementations, data repositories 730 can include a database or another type of data storage system or framework for organizing and storing data. In some implementations, DICOM servers 720 and data repository 730 can be connected via network 750. Platform management terminal 740 can include any type of wired or wireless user device capable of communicating with DICOM servers 720 and/or data repositories 730 via network 750. Platform management terminal 740 can include a smartphone, tablet computer, laptop computer, desktop computer, or another type of user device capable of enabling a user, operator, administrator, or developer to interact with DICOM servers 720 and/or the DICOM platform. In some implementations, platform management terminal 740 can be a UE 710. Additionally, or alternatively, platform management terminal 740 can be directly connected to DICOM servers 720. In some implementations, data repositories 730 can be, or can be part of, a VNA system.

DICOM sources/destinations 760 can include one or more computing devices, such as user devices, network devices, or server device capable of receiving, processing, storing, and communicating information via network 750. DICOM sources/destinations 760 can be owned or operated by a particular institution (e.g., a doctor's office, hospital, research institution, government agency, or records archive). DICOM sources/destinations 760 can be capable of creating and sending DICOM information (e.g., DICOM studies or DICOM objects) to DICOM servers 720 for processing and/or storage. Additionally, or alternatively, DICOM sources/destinations 750 can request and receive DICOM information from DICOM servers 720 and/or form another DICOM source/destination. In some implementations, DICOM sources/destinations 760 a VNA system capable of receiving, storing, and distributing DICOM information to DICOM servers 720 and/or other DICOM sources/destinations 760.

Network 750 can include a single network or multiple networks capable of enabling a connection between the devices of FIG. 7. Network 750 can include one or more wired and/or wireless networks. For example, network 750 can include a Bluetooth® network, a Wi-Fi network, or a cellular network, the Public Land Mobile Network (PLMN), and/or a second generation (2G) network, a third generation (3G) network, a fourth generation (4G) network, a fifth generation (5G) network, a sixth generation (6G) network and/or another type of network. Additionally, or alternatively, network 750 can include a wide area network (WAN), a metropolitan area network (MAN), an ad hoc network, an intranet, the Internet, a virtual network (e.g., a virtual private network (VPN)), a telephone network (e.g., a Public Switched Telephone Network (PSTN)), a Voice over IP (VOIP) network, and/or a combination of these or other types of networks.

In various implementations or aspects of this disclosure, the DICOM servers 720 can access the XDS profile of the system 200 to perform morphing of XDS content or documents, metadata or attributes as described. The data repository 730 can operate as a second set of data repositories for XDS content or content of another formatting (e.g., DICOM, HL7, or the like). Any one or more devices, components or networks can operate as the document consumer 108 or communicate and interact with the document consumer 108. Likewise, the morphing component 202 can be located in or operable from any one of the devices, components or networks of FIG. 7 or FIG. 2, for example.

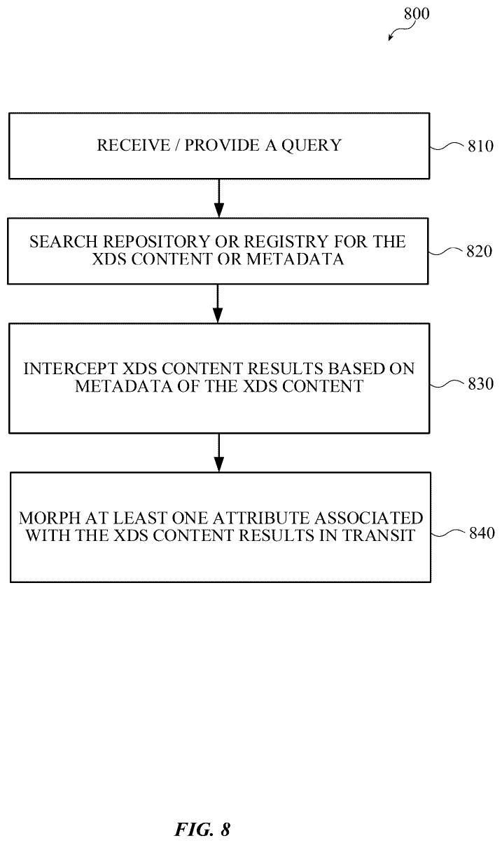

FIG. 8 illustrates an example process flow 800 for morphing XDS documents of a healthcare enterprise system. The process flow 800 can initiate at 810 by receiving a query. The query can be for XDS content in a first document repository or registry (e.g., document repository 104 or document registry 106). At 820, the process flow 800 includes searching the first document repository for the XDS content based on criteria of the query. At 830, the process flow 800 includes intercepting XDS content results based on metadata of the XDS content satisfying the query before being provided in response to the query. At 840, the process flow 800 includes morphing at least one attribute (e.g., a study description or numeric value) associated with the XDS content results in transit before providing the XDS content results to a graphical user interface (e.g., UE 710) or a second document repository (e.g., data repository 730).

The process flow 800 can further include updating a string of a study description to provide the XDS content results based on an updated string. The updated string can then be associated with an EventCode for use with the second document repository.

The process flow 800 can further remove at least a portion of a string from a study description of the at least one attribute before the XDS content results are provided to the graphical user interface or the second document repository in response to the query. The second document repository can comprises a content of a different format than the XDS content in the first document repository, or be a same format with a different or incorrect value or alpha-numeric character, or with a different pattern, which is to be morphed for an update.

The process flow 800 can include determining a patient ID comprising the attribute associated with the metadata of the XDS content. Non-significant digits (e.g., zeros) that satisfy a predetermined length or length threshold of a patient ID can be removed or added, for example. The patient ID that is associated with the metadata of the XDS content in the XDS content results can be modified to an updated patient ID for the second document repository.

The process flow 800 can include mapping a patient ID associated with the metadata of the XDS content results of the first document repository or the second document repository with one or more other patient IDs associated with metadata of a second set of content comprising at least one of: the XDS content or DICOM content associated with a same patient information. One or more patient IDs comprising a local patient ID associated with the second document repository can be mapped to a patient ID associated with the first or the second document repository. The process flow 800 can include searching the first document for the XDS content based on the local patient ID in response to receiving the query.

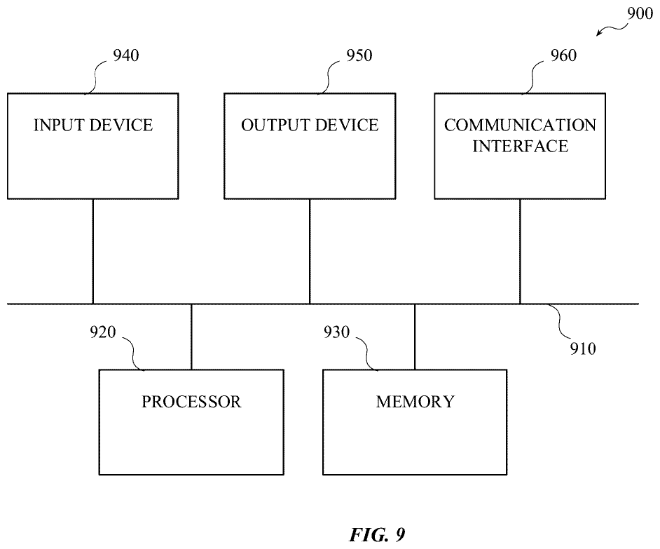

FIG. 9 is a diagram of example components of a device 900 that can be used with/within environment or systems described in reference to FIGS. 1 thru 8. Device 900 can correspond to UE 710, DICOM servers 720, data repository 730, platform management terminal 740, and/or DICOM information source/destination 760. Each of UE 710, DICOM servers 720, data repository 730, platform management terminal 740, and/or DICOM information source/destination 760 can include one or more of devices 900 and/or one or more of the components of device 900, for example, as with the components of other figures herein.

As depicted, device 900 can include bus 910, processor 920, memory 930, input device 940, output device 950, and communication interface 960. However, the precise components of device 900 can vary between implementations. For example, depending on the implementation, device 900 can include fewer components, additional components, different components, or differently arranged components than those illustrated in FIG. 9.

Bus 910 can permit communication among the components of device 900. Processor 920 can include one or more processors, microprocessors, data processors, co-processors, network processors, application-specific integrated circuits (ASICs), controllers, programmable logic devices (PLDs), chipsets, field-programmable gate arrays (FPGAs), or other components that can interpret or execute instructions or data. Processor 920 can control the overall operation, or a portion thereof, of device 900, based on, for example, an operating system (not illustrated), and/or various applications. Processor 920 can access instructions from memory 930, from other components of device 900, or from a source external to device 900 (e.g., a network or another device).

Memory 930 can include memory and/or secondary storage. For example, memory 930 can include random access memory (RAM), dynamic RAM (DRAM), read-only memory (ROM), programmable ROM (PROM), flash memory, or some other type of memory. Memory 930 can include a hard disk (e.g., a magnetic disk, an optical disk, a magneto-optic disk, or a solid state disk) or some other type of computer-readable medium, along with a corresponding drive. A computer-readable medium can be defined as a non-transitory memory device. A memory device can include space within a single physical memory device or spread across multiple physical memory devices.

Input device 940 can include one or more components that permit a user to input information into device 900. For example, input device 940 can include a keypad, a button, a switch, a knob, fingerprint recognition logic, retinal scan logic, a web cam, voice recognition logic, a touchpad, an input port, a microphone, a display, or some other type of input component. Output device 950 can include one or more components that permit device 900 to output information to a user. For example, output device 950 can include a display, light-emitting diodes (LEDs), an output port, a speaker, or some other type of output component.

Communication interface 960 can include one or more components that permit device 900 to communicate with other devices or networks. For example, communication interface 960 can include some type of wireless or wired interface. Communication interface 960 can also include an antenna (or a set of antennas) that permit wireless communication, such as the transmission and reception of radio frequency (RF) signals.

As described herein, device 900 can perform certain operations in response to processor 920 executing software instructions contained in a computer-readable medium, such as memory 930. The software instructions can be read into memory 930 from another computer-readable medium or from another device via communication interface 960. The software instructions contained in memory 930 can cause processor 920 to perform one or more processes described herein. Alternatively, hardwired circuitry can be used in place of, or in combination with, software instructions to implement processes described herein. Thus, implementations described herein are not limited to any specific combination of hardware circuitry and software.

Examples herein can include subject matter such as a method, means for performing acts or blocks of the method, at least one machine-readable medium including executable instructions that, when performed by a machine (e.g., a processor) with memory, an application-specific integrated circuit (ASIC), a field programmable gate array (FPGA), or the like) cause the machine to perform acts of the method or of an apparatus or system for concurrent communication using multiple communication technologies according to implementations and examples described.

The above description of illustrated examples, implementations, aspects, etc., of the subject disclosure, including what is described in the Abstract, is not intended to be exhaustive or to limit the disclosed aspects to the precise forms disclosed. While specific examples, implementations, aspects, etc., are described herein for illustrative purposes, various modifications are possible that are considered within the scope of such examples, implementations, aspects, etc., as those skilled in the relevant art can recognize.

In this regard, while the disclosed subject matter has been described in connection with various examples, implementations, aspects, etc., and corresponding Figures, where applicable, it is to be understood that other similar aspects can be used or modifications and additions can be made to the disclosed subject matter for performing the same, similar, alternative, or substitute function of the subject matter without deviating therefrom. Therefore, the disclosed subject matter should not be limited to any single example, implementation, or aspect described herein, but rather should be construed in breadth and scope in accordance with the appended claims below.

In particular regard to the various functions performed by the above described components or structures (assemblies, devices, circuits, systems, etc.), the terms (including a reference to a “means”) used to describe such components are intended to correspond, unless otherwise indicated, to any component or structure which performs the specified function of the described component (e.g., that is functionally equivalent), even though not structurally equivalent to the disclosed structure which performs the function in the herein illustrated exemplary implementations. In addition, while a particular feature can have been disclosed with respect to only one of several implementations, such feature can be combined with one or more other features of the other implementations as can be desired and advantageous for any given application.

As used herein, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form. Furthermore, to the extent that the terms “including”, “includes”, “having”, “has”, “with”, or variants thereof are used in either the detailed description or the claims, such terms are intended to be inclusive in a manner similar to the term “comprising.” Additionally, in situations wherein one or more numbered items are discussed (e.g., a “first X”, a “second X”, etc.), in general the one or more numbered items can be distinct, or they can be the same, although in some situations the context can indicate that they are distinct or that they are the same.

It is well understood that the use of personally identifiable information should follow privacy policies and practices that are generally recognized as meeting or exceeding industry or governmental requirements for maintaining the privacy of users. In particular, personally identifiable information data should be managed and handled to minimize risks of unintentional or unauthorized access or use, and the nature of authorized use should be clearly indicated to users.

What is claimed is:

1. A non-transitory computer-readable medium comprising:
instructions that when executed by one or more processors, cause the one or more processors to:
receive a query for Cross-Enterprise Document Sharing (XDS) content in a first document repository;
search the first document repository for the XDS content based on the query;
intercept XDS content results based on metadata of the XDS content satisfying the query before being provided in response to the query; and
morph at least one attribute associated with the XDS content results in transit before providing the XDS content results to a graphical user interface or a second document repository.

2. The non-transitory computer-readable medium of claim 1, wherein the at least one attribute comprises a study description, and the instructions further cause the one or more processors to:
update a string of the study description to provide the XDS content results based on an updated string; and
associate the updated string with an EventCode for use with the second document repository, wherein the second document repository stores content that is in a different format than the XDS content.

3. The non-transitory computer-readable medium of claim 1, wherein the instructions further cause the one or more processors to:
remove at least a portion of a string from a study description of the at least one attribute before the XDS content results are provided to the graphical user interface or the second document repository in response to the query, wherein the second document repository comprises a content of a different format than the XDS content in the first document repository.

4. The non-transitory computer-readable medium of claim 1, wherein the second document repository stores content of a different format than the XDS content, the content of a different format including one or more key object selection (KOS) Digital Imaging and Communications in Medicine (DICOM) documents.

5. The non-transitory computer-readable medium of claim 1, wherein the instructions further cause the one or more processors to:
determine a patient ID comprising the attribute associated with the metadata of the XDS content;
remove non-significant digits of the patient ID; and
modify the patient ID that is associated with the metadata of the XDS content in the XDS content results to an updated patient ID for the second document repository.

6. The non-transitory computer-readable medium of claim 5, wherein the non-significant digits comprise zeros in the patient ID to satisfy a predetermined length.

7. The non-transitory computer-readable medium of claim 1, wherein the instructions further cause the one or more processors to:
map a patient ID associated with the metadata of the XDS content results of the first document repository or the second document repository with one or more other patient IDs associated with metadata of a second set of content comprising at least one of: the XDS content or DICOM content associated with a same patient information.

8. The non-transitory computer-readable medium of claim 1, wherein the instructions further cause the one or more processors to:
map one or more patient IDs comprising a local patient ID associated with the second document repository to a patient ID associated with the first document repository, wherein the second document repository comprises content in a different format than the XDS content of the first document repository; and
in response to receiving the query, search the first document for the XDS content based on the local patient ID.

9. A system of a healthcare enterprise comprising:
a Cross-Enterprise Document Sharing (XDS) registry comprising a registry input/output for metadata of XDS content in a first document repository, and configured to control metadata that comprises attributes of a medical document or image; and
a patient manager comprising a query input/output communicatively coupled to the registry input/output, and configured to transform the attributes of the metadata associated with the XDS content in response to a query and before providing an XDS content result of the query to a graphical user interface or a second document repository based on a set of XDS rules to be performed.

10. The system of claim 9, wherein the set of XDS rules comprise transforming a patient ID of the XDS content between a first patient ID format from an XDS content format of the first document repository and a second patient ID format of the second document repository.

11. The system of claim 10, wherein the transforming comprises performing a removal of non-significant digits of the patient ID of the XDS content based on a predetermined length and whether the patient ID is being transmitted or received by the XDS registry.

12. The system of claim 9, wherein the set of XDS rules comprise mapping one or more different patient IDs from a different healthcare enterprise to at least one patient ID associated with the attributes or the metadata of the XDS content.

13. The system of claim 12, wherein the patient manager determines whether a patient ID is mapped to the one or more different patient IDs, and in response to the patient ID being mapped to the one or more different patient IDs, queries the XDS content and content of a different format of the different healthcare enterprise with any one of the patient ID and the one or more different patient IDs.

14. The system of claim 9, wherein the set of XDS rules comprise transforming XDS content request metadata or XDS content response metadata for a query by intercepting the metadata of the XDS content associated with the XDS content request metadata or the XDS content response metadata and modifying a portion of a string from a study description of the metadata of the XDS content.

15. The system of claim 9, wherein the set of XDS rules comprises a set of predefined actions to the metadata of the XDS content based on as selection among the set of predefined actions and based on whether the metadata of the XDS content is in response to an XDS request or an XDS response before being stored in the first document repository or the second document repository.

16. A method of a healthcare enterprise comprising:
storing, in a first document repository via processing circuitry, Cross-Enterprise Document Sharing (XDS) content associated with a patient ID;
storing, in a second document repository, a second set of content comprising at least one of: XDS content or DICOM content associated with a patient information of the XDS content;
receiving a query for metadata of at least one of the first document repository or the second document repository;
searching the at least one of the first document repository or the second document repository based on the query to obtain XDS content results with the metadata satisfying the query;
intercepting the XDS content results before being provided to the second document repository in response to the query; and
morphing at least one attribute of the metadata of the XDS content in transit before providing the XDS content results to a graphical user interface or the second document repository.

17. The method of claim 16, further comprising:
intercepting the query with an enterprise master patient index (EMPI) source by at least one of the patient ID or attributes of the patient information to obtain the patient information; and
mapping one or more different patient IDs from a different healthcare enterprise to the patient ID or to the attributes of the patient information in at least one of the first document repository or the second document repository.

18. The method of claim 16, wherein the morphing the at least one attribute of the metadata of the XDS content includes satisfying a set of XDS rules for converting the at least one attribute to a different format, wherein the set of XDS rules include a modification of a string of the metadata, and a customizable rule that includes a dynamic link library (DLL) to provide user selected functions for the morphing of the at least one attribute of the metadata of the XDS content in transit.

19. The method of claim 16, wherein the morphing includes transforming an XDS simple object access protocol (SOAP) request or response in extensible mark-up language (XML) with a user customizable extensible style sheet language transformation (XSLT) code.

20. The method of claim 16, further comprising:
transferring the patient information from a hospital information system (HIS) database or a radio information system (RIS) database of another healthcare enterprise while morphing the patient information based on whether the patient information is incoming or outgoing from at least one of the first document repository or the second document repository.
